(19) United States
(12) Patent Application Publication
(10) Pub. No.: US 2011/0263718 A1
(43) Pub. Date: Oct. 27, 2011

(54) PROCESS FOR THE PREPARATION OF O-DESMETHYLVENLAFAXINE

(75) Inventors: Vinayak Gore, Maharashtra (IN); Vinay Kumar Shukla, Maharashtra (IN); Madhukar Patil, Maharashtra (IN)

(73) Assignee: Generics [UK] Limited, Hertfdordshire (GB)

(21) Appl. No.: 13/056,025

(22) PCT Filed: Jul. 29, 2009
(86) PCT No.: PCT/GB2009/050943

§ 371 Date: Jul. 12, 2011

(30) Foreign Application Priority Data
Jul. 30, 2008 (IN) 1288/KOL/2008

(51) Int. Cl. A61K 31/137; A61P 25/24; A61P 25/22; A61P 25/00; A61P 25/18; A61P 25/16; A61P 9/00; A61P 25/30; A61P 25/32; A61P 15/00; A61P 13/00; C07C 211/27; A61P 3/04

(52) U.S. Cl. 514/654; 564/374

(57) ABSTRACT

The present invention provides a convenient and efficient process for the preparation of O-desmethylvenlafaxine (ODV) or a salt thereof, comprising the reaction of venlafaxine, or a salt thereof, with a thiourea or a mixture of thioureas.

### FIELD OF THE INVENTION

The present invention provides a convenient and efficient process for the preparation of O-desmethylvenlafaxine (ODV) or a salt thereof, comprising the reaction of venlafaxine, or a salt thereof, with a thiourea or a mixture of thioureas.

### BACKGROUND OF THE INVENTION

O-Desmethylvenlafaxine (ODV, II), chemically named 1-[1-(4-hydroxyphenyl)-2-(dimethylamino)ethyl]cyclohexanol, is a major metabolite of venlafaxine. ODV is known to inhibit norepinephrine and serotonin uptake and to have antidepressant activity. It has been further reported that oral administration of ODV succinate, in particular sustained release oral administration of ODV succinate, results in a lower incidence of nausea, vomiting, diarrhea, abdominal pain, headache, vaso-vagal malaise and/or trismus than oral administration of venlafaxine. ODV is known to be effective in treating patients suffering from depression, anxiety and panic disorder.

Various prior art patents and patent applications describe processes for the preparation of ODV free base, which can be converted into a desired pharmaceutically acceptable salt. Such prior art processes to obtain ODV are disclosed in documents U.S. Pat. No. 4,535,186, U.S. Pat. No. 6,673,838, U.S. Pat. No. 4,761,501, WO 03/48104, WO 00/59851, WO 00/32556, WO 00/76955, WO 00/32555, WO 02/64543, WO 2007/071404 and U.S. Pat. No. 4,761,501.

The process described in U.S. Pat. No. 4,535,186 for the preparation of ODV leads to relatively low yields and throughput as benzyl protecting groups are used.

Other prior art patents listed above describe processes for making ODV which avoid using protecting groups as demethylation of venlafaxine is used instead (Scheme 1). However, in general, the substituted phenoxy group of venlafaxine is a very stable moiety and thus the demethylation reaction typically requires special reagents and drastic conditions. Furthermore, the reagent must be carefully selected so that it does not attack the tertiary hydroxy group on the cyclohexane ring on venlafaxine. The starting material, venlafaxine or a salt thereof, may be prepared in accordance with procedures known in the art, such as in U.S. Pat. No. 4,535,186.

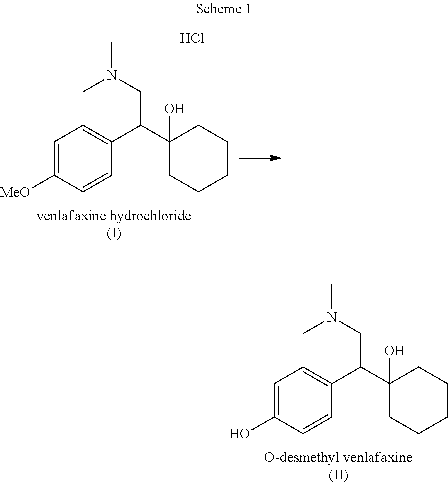

WO 00/59851, WO 00/32556 and WO 00/32555 disclose a process for preparing ODV starting from venlafaxine using lithium diphenyl phosphide (prepared in situ from diphenyl phosphine and n-butyl lithium) as the demethylation agent and tetrahydrofuran as a solvent. However, disadvantages of this process are that the concentration of the material in the solvent is very low and the presence of a largely insoluble lithium salt of venlafaxine which is formed in the tetrahydrofuran solvent.

WO 02/64543 discloses a process for the preparation of ODV by demethylation of venlafaxine using reagents such as L-selectride. However, this process is relatively expensive due to the cost of the reagents.

Processes are also disclosed, describing demethylation using boron tribromide as the reagent. However, this process suffers from the major disadvantages of the requirement of low temperature and the hazards involved in the use of boron tribromide. Consequently, this process is not amenable to large scale.

WO 02/64543 and WO 03/48104 disclose a demethylation process using the sodium salt of dodecane thiol in polyethylene glycol 400 at 190-200° C. This process suffers from the disadvantage that the decomposition of ODV is unavoidable at such high temperatures. In addition, there is the need to employ two solvents: methanol for formation of a suspension of sodium methoxide and then polyethylene glycol 400 to run the reaction at high temperature. This necessitates removal of methanol from the reaction mixture to attain high reaction temperatures and to drive the reaction to completion.

WO 00/76955 discloses a demethylation process using the sodium salt of ethane thiol, however, this process suffers from the disadvantages of not being very high yielding and affording a product of low purity. The use of the low boiling ethane thiol (b.p. 35° C.) means that handling and storage of the reagent on an industrial scale is difficult and has safety problems. In addition, ethane thiol is very toxic and has a very noxious smell which is also not suitable for industrial manufacture. In addition, the use of sodium hydride to form the sodium salt of ethane thiol is also not convenient on a commercial scale.

WO 2007/071404 discloses the use of sodium sulphide as the reagent for demethylation of venlafaxine. However, the process has the disadvantage of requiring an inconvenient, prolonged reaction time of around 30 hours.

Thus, the processes disclosed in the prior art suffer from several disadvantages such as moderate to low yields; obtaining ODV (II) in an impure state; very high temperatures; lengthy processes; and/or using expensive, toxic and/or hazardous reagents, which are not recommended to be used on a commercial scale, such as L-selectride, ethane thiol, boron tribromide and n-butyl lithium.

Therefore, it would be desirable to develop an alternative, improved process for the demethylation of venlafaxine to obtain ODV, which process is efficient, non-hazardous and economical.

### OBJECT OF THE INVENTION

An object of the present invention is to provide a new, efficient, non-hazardous and economical process for converting venlafaxine into ODV by demethylation.

### DEFINITIONS

For the purposes of the present invention, an “alkyl” group is defined as a monovalent saturated hydrocarbon, which may be straight-chained or branched, or be or include cyclic groups. An alkyl group may optionally be substituted, and may optionally include one or more heteroatoms N, O or S in its carbon skeleton. Preferably an alkyl group is straight-chained or branched. Preferably an alkyl group is not substituted. Preferably an alkyl group does not include any heteroatoms in its carbon skeleton. Examples of alkyl groups are methyl, ethyl, n-propyl, i-propyl, n-butyl, i-butyl, t-butyl, n-pentyl, cyclopentyl, cyclohexyl and cycloheptyl groups. Preferably an alkyl group is a C₁₋₂ alkyl group, which is defined as an alkyl group containing from 1 to 12 carbon atoms. More preferably an alkyl group is a C₁₋₆ alkyl group. Preferably a cyclic alkyl group is a C₃₋₁₂ cyclic alkyl group, preferably a C₅₋₇ cyclic alkyl group. An “alkylene” group is similarly defined as a divalent alkyl group.

As used herein, the term “alkoxide” means alkyl-O⁻.

An “alkenyl” group is defined as a monovalent hydrocarbon, which comprises at least one carbon-carbon double bond, which may be straight-chained or branched, or be or include cyclic groups. An alkenyl group may optionally be substituted, and may optionally include one or more heteroatoms N, O or S in its carbon skeleton. Preferably an alkenyl group is straight-chained or branched. Preferably an alkenyl group is not substituted. Preferably an alkenyl group does not include any heteroatoms in its carbon skeleton. Examples of alkenyl groups are vinyl, allyl, but-1-enyl, but-2-enyl, cyclohexenyl and cycloheptenyl groups. Preferably an alkenyl group is a C₂₋₁₂ alkenyl group, preferably a C₂₋₆ alkenyl group. Preferably a cyclic alkenyl group is a C₃₋₁₂ cyclic alkenyl group, preferably a C₅₋₇ cyclic alkenyl group. An “alkenylene” group is similarly defined as a divalent alkenyl group.

An “alkynyl” group is defined as a monovalent hydrocarbon, which comprises at least one carbon-carbon triple bond, which may be straight-chained or branched, or be or include cyclic groups. An alkynyl group may optionally be substituted, and may optionally include one or more heteroatoms N, O or S in its carbon skeleton. Preferably an alkynyl group is straight-chained or branched. Preferably an alkynyl group is not substituted. Preferably an alkynyl group does not include any heteroatoms in its carbon skeleton. Examples of alkynyl groups are ethynyl, propargyl, but-1-ynyl and but-2-ynyl groups. Preferably an alkynyl group is a C₂₋₁₂ alkynyl group, preferably a C₂₋₆ alkynyl group. Preferably a cyclic alkynyl group is a C₃₋₁₂ cyclic alkynyl group, preferably a C₅₋₇ cyclic alkynyl group. An “alkynylene” group is similarly defined as a divalent alkynyl group.

An “aryl” group is defined as a monovalent aromatic hydrocarbon. An aryl group may optionally be substituted, and may optionally include one or more heteroatoms N, O or S in its carbon skeleton. Preferably an aryl group is unsubstituted or mono-substituted. Preferably an aryl group does not include any heteroatoms in its carbon skeleton. Examples of aryl groups are phenyl, naphthyl, anthracenyl and phenanthrenyl groups. Preferably an aryl group is a C₄₋₁₄ aryl group, preferably a C₆₋₁₀ aryl group. An “arylene” group is similarly defined as a divalent aryl group.

For the purposes of the present invention, where a combination of groups is referred to as one moiety, for example, arylalkyl, arylalkenyl, arylalkynyl, alkylaryl, alkenylaryl or alkynylaryl, the last mentioned group contains the atom by which the moiety is attached to the rest of the molecule. A typical example of an arylalkyl group is benzyl.

For the purposes of this invention, an optionally substituted hydrocarbon or alkyl, alkenyl, alkynyl, aryl, arylallyl, arylalkenyl, arylalkynyl, alkylaryl, alkenylaryl or alkynylaryl group may be substituted with one or more of —F, —Cl, —Br, —I, —CF₃, —CCl₃, —CBr₃, —Cl₃, —OH, —SH, —NH₂, —CN, —NO₂, —COOH, —R^{α}—O—R^{β}, —R^{α}—S—R^{β}, —R^{α}—SO—R^{β}, —R^{α}SO₂—R^{β}, —R^{β}—SO₂—OR^{β}, —R^{α}O—SO₂—R^{β}, —R^{α}—SO₂—N(R^{β})₂, —R^{α}—NR^{β}—SO₂—R^{β}, —R^{α}O—SO₂—OR^{β}, —R^{α}O—SO₂—N(R^{β})₂, —R^{α}—NR^{β}—SO₂—OR^{β}, —R^{α}—NR^{β}—SO₂—N(R^{β})₂, —R^{α}N(R^{β})₂, —R^{α}N(R^{β})₃⁺, —R^{β}—P(R^{β})₂, —R^{α}—Si(R^{β})₃, —R^{α}—CO—R^{β}, —R^{α}—CO—OR^{β}, —R^{α}—CO—R^{β}, —R^{α}—CO—N(R^{β})₂, —R^{α}—NR^{β}—CO—R^{β}, —R^{α}O—CO—OR^{β}, —R^{α}O—CO—N(R^{β})₂, —R^{α}—NR^{β}—CO—OR^{β}, —R^{α}—NR^{β}—CO—N(R^{β})₂, —R^{α}—CS—R^{β}, —R^{α}—CS—OR^{β}, —R^{α}O—CS—R^{β}, —R^{α}—CS—N(R^{β})₂, —R^{α}—NR^{β}—CS—R^{β}, —R^{α}O—CS—OR^{β}, —R^{α}O—CS—N(R^{β})₂, —R^{α}—NR^{β}—CS—OR^{β}, —R^{α}—NR^{β}—CS—N(R^{β})₂, —R^{β}, a bridging substituent such as —O—, —S—, —NR^{β}— or —R^{α}, or a π-bonded substituent such as ═O, ═S or ═NR^{β}. In this context, —R^{α}— is independently a chemical bond, a C₁-C₁₀ alkylene, C₂-C₁₀ alkenylene or C₂-C₁₀ alkynylene group. —R^{β} is independently hydrogen, unsubstituted C₁-C₆ alkyl or unsubstituted C₆-C₁₀ aryl. Optional substituent(s) are preferably taken into account when calculating the total number of carbon atoms in the parent group substituted with the optional substituent(s). Preferably an optionally substituted hydrocarbon or alkyl, alkenyl, alkynyl, aryl, arylalkyl, arylalkenyl, arylalkynyl, alkylaryl, alkenylaryl or alkynylaryl group is not substituted with a bridging substituent. Preferably an optionally substituted hydrocarbon or alkyl, alkenyl, alkynyl, aryl, arylallyl, arylalkenyl, arylalkynyl, alkylaryl, alkenylaryl or alkynylaryl group is not substituted with a π-bonded substituent. Preferably a substituted group comprises 1, 2 or 3 substituents, more preferably 1 or 2 substituents, and even more preferably 1 substituent.

Any optional substituent may be protected. Suitable protecting groups for protecting optional substituents are known in the art, for example from “Protective Groups in Organic Synthesis” by T. W. Greene and P. G. M. Wuts (Wiley-Interscience, 4^{th} edition, 2006).

Where appropriate, the compounds of the present invention can be used both, in their free base form and their acid addition salt form. For the purposes of this invention, a “salt” of a compound of the present invention encompasses an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulphuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulphonic acids (for example, methanesulphonic, trifluoromethanesulphonic, ethanesulphonic, 2-hydroxyethanesulphonic, benzenesulphonic, toluene-p-sulphonic, naphthalene-2-sulphonic or camphorsulphonic acid) or amino acids (for example, ornithinic, glutamic or aspartic acid). The acid addition salt may be a mono- or di-acid addition salt, preferably a mono-acid addition salt. A preferred salt is a hydrohalogenic, sulphuric, phosphoric or organic acid addition salt. More preferred salts are succinic, fumaric and hydrochloric acid addition salts.

In addition to pharmaceutically acceptable acid addition salts, other acid addition salts are included in the present invention, since they have potential to serve as intermediates in the purification or preparation of other, for example, pharmaceutically acceptable, acid addition salts, or are useful for identification, characterisation or purification of the free base.

Where appropriate, the compounds of the present invention can be used both, in their free acid form and their salt form. For the purposes of this invention, a “salt” of a compound of the present invention encompasses those formed between of a compound of the present invention, such as a thiourea anion, and a suitable cation. Suitable cations include, but are not limited to lithium, sodium, potassium, magnesium, calcium and ammonium. The salt may be a mono-, di- or tri-salt. Preferably the salt is a mono- or di-lithium, sodium, potassium, magnesium, calcium or ammonium salt. More preferably the salt is a mono-sodium salt. Preferably the salt is a pharmaceutically acceptable salt.

The present invention encompasses pharmaceutically acceptable salts, derivatives, solvates, clathrates and/or hydrates (including anhydrous forms) of the compounds of the present invention.

### SUMMARY OF THE INVENTION

According to the first aspect of the invention there is provided a process for the preparation of O-desmethylvenlafaxine (ODV, II), or a salt such as a pharmaceutically acceptable salt thereof, comprising the reaction of venlafaxine, or a salt thereof, with a thiourea or a mixture of thioureas.

The term “thiourea” as used herein throughout the description and claims includes salts thereof and can mean thiourea (III) or a substituted thiourea (IV), wherein R¹, R², R³ and R⁴ can be independently hydrogen, alkyl, alkenyl, allynyl, aryl, arylalkyl, arylalkenyl, arylalkynyl, alkylaryl, alkenylaryl or alkynylaryl. Most preferably the reagent is thiourea (III) which is a readily available low molecular weight compound.

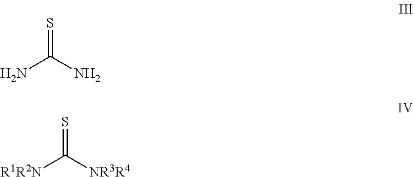

Preferably when the thiourea is a substituted thiourea (IV), at least one of R¹, R², R³ or R⁴ is hydrogen. Preferably where R¹, R², R³ or R⁴ is an alkyl, alkenyl, alkynyl, aryl, arylalkyl, arylalkenyl, arylalkynyl, alkylaryl, alkenylaryl or alkynylaryl group, the atom by which the group is attached to the nitrogen atom of the remainder of the thiourea is not substituted with a π-bonded substituent and/or is not a heteroatom N, O or S.

Preferably the process according to the first aspect of the invention is carried out in a reaction solvent. The reaction solvent is preferably selected from an alcohol, ethylene glycol, an ether of ethylene glycol or a mixture thereof, such as polyethylene glycol (e.g. polyethylene glycol 400), cellosolve or 1-butanol.

Preferably the reaction solvent is a single solvent. Preferably the reaction solvent has a boiling point of at least 100° C., more preferably at least 120° C., more preferably at least 140° C., most preferably at least 160° C. Suitable solvents include for instance toluene, chlorobenzene, 1-butanol, ethylene glycol, di-n-butyl ether, 1,4-dioxane, mono- and di-ethers of ethylene glycol such as cellosolve, polyethylene glycols such as polyethylene glycol 400, γ-butyrolactone, propylene carbonate, aniline, benzonitrile, pyridine, N,N-dimethylformamide, N,N-dimethylacetamide, N,N-dimethylpropyleneurea, nitrobenzene, hexamethylphosphoramide, tetramethylurea, dimethylsulphoxide and sulpholane. Preferred solvents include ethylene glycol, polyethylene glycols such as polyethylene glycol 400, γ-butyrolactone, propylene carbonate, aniline, benzonitrile, N,N-dimethylformamide, N,N-dimethylacetamide, N,N-dimethylpropyleneurea, nitrobenzene, hexamethylphosphoramide, tetramethylurea, dimethylsulphoxide and sulpholane. Most preferably the solvent is selected from ethylene glycol or polyethylene glycols such as polyethylene glycol 400.

Preferably a base is used to generate an anion of the thiourea reagent, to facilitate the reaction in the process according to the first aspect of the invention. The base is preferably a monovalent or bivalent metal hydroxide, carbonate, hydrogen carbonate or alkoxide. Alternatively, organic bases, such as alkyl or aryl amines (for example piperidine or pyridine) may be used. Where the base is an organic base such as aniline or pyridine, it may in addition act as the reaction solvent. Preferably the base is a metal hydroxide or metal alkoxide. Most preferably, the base is potassium hydroxide or sodium methoxide. The thiourea anion is preferably prepared in situ in the same solvent used for running the reaction.

Preferably the process according to the first aspect of the invention is performed at a temperature within the range of 100-220° C., more preferably within the range of 130-180° C., and most preferably within the range of 160-180° C.

Preferably the reaction of venlafaxine, or a salt thereof, with the thiourea or the mixture of thioureas is carried out for 10-24 hours, preferably for 16-20 hours.

Preferably, to remove process impurities during the work up procedure of the process according to the first aspect of the invention, the reaction mixture is washed with a solvent that is immiscible with water at 20° C. and 1 atmosphere pressure. Suitable solvents include for instance hydrocarbon solvents, such as hexane, heptane, cyclohexane, toluene, xylene or mixtures thereof, ethers such as diethyl ether, diisopropyl ether or mixtures thereof, esters such as ethyl acetate, or halogenated hydrocarbon solvents, such as dichloromethane, ethylene dichloride or mixtures thereof.

Preferably the product formed in the process according to the first aspect of the invention is purified by crystallization with an alcohol to generate a product with high purity. Preferably the alcohol is monohydric. Preferably the alcohol is a C₁-C₆ alcohol, more preferably the alcohol is a C₁-C₄ alcohol such as one selected from methanol, ethanol, isopropanol or a mixture thereof, more preferably the alcohol is methanol.

Alternatively or additionally, the product formed in the process according to the first aspect of the invention is preferably purified by mixing with an alcohol, such as methanol, ethanol or isopropanol or a mixture thereof, to form a suspension and then adding acid followed by base to generate ODV base with high purity. Most preferably, the alcohol is methanol. Preferably the acid used is an inorganic acid such as hydrochloric acid or sulphuric acid. Preferably the base used is an organic base such as triethylamine or trimethylamine. Alternatively, the base used can be an inorganic base such as ammonia, sodium carbonate, potassium carbonate or sodium hydroxide.

Preferably the process according to the first aspect of the invention is carried out on an industrial scale, preferably to obtain batches of ODV base, or a salt such as a pharmaceutically acceptable salt thereof, of 100 g, 500 g, 1 kg, 5 kg, 10 kg, 50 kg, 100 kg or more.

Preferably the ODV base prepared by the process according to the first aspect of the invention is at least 95% pure, at least 98% pure, at least 99% pure, at least 99.5% pure, or at least 99.9% pure. Preferably the purity is as analysed by HPLC.

Preferably the ODV base prepared by the process according to the first aspect of the invention is obtained in a yield of 25% or more, preferably 30% or more, preferably 50% or more, preferably 60% or more, preferably 70% or more, preferably 80% or more, preferably 85% or more.

Preferably the pharmaceutically acceptable salt of ODV prepared by the process according to the first aspect of the invention is selected from the succinate or fumarate salt. Preferably the salt of venlafaxine used in the process according to the first aspect of the invention is the hydrochloride salt.

In a second aspect of the present invention, there is provided ODV or a salt such as a pharmaceutically acceptable salt thereof prepared by a process according to the first aspect of the invention. Preferred salts of the second aspect of the invention are the succinate and fumarate salts. Preferably the ODV or the salt thereof of the second aspect of the invention is suitable for use in medicine, preferably for treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's Disease.

In a third aspect of the present invention, there is provided a pharmaceutical composition comprising ODV or a pharmaceutically acceptable salt thereof prepared by a process according to the first aspect of the invention. Preferably the pharmaceutical composition according to the third aspect of the invention comprises ODV succinate or ODV fumarate.

Preferably the pharmaceutical composition comprises one or more conventional pharmaceutically acceptable excipient(s). Preferably the pharmaceutical composition according to the third aspect of the invention is suitable for treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's Disease.

In a fourth aspect of the present invention, there is provided the use of the ODV or the pharmaceutically acceptable salt thereof according to the second aspect of the invention, or the use of the pharmaceutical composition according to the third aspect of the invention, for the preparation of a medicament for the treatment or prevention of depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's Disease.

In a fifth aspect of the present invention, there is provided a method of treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's Disease, the method comprising administering to a patient in need thereof a therapeutically or prophylactically effective amount of the ODV or the pharmaceutically acceptable salt thereof according to the second aspect of the invention, or a therapeutically or prophylactically effective amount of the pharmaceutical composition according to the third aspect of the invention. Preferably the patient is a mammal, preferably a human.

### DETAILED DESCRIPTION OF THE INVENTION

ODV base and its salts exist as enantiomers and the present invention includes racemic mixtures as well as stereoisomerically pure forms of the same. The term “ODV” as used herein refers to the racemic mixtures and stereoisomerically pure forms of ODV, unless otherwise indicated. The term “stereoisomerically pure” refers to compounds, which are comprised of a greater proportion of the desired isomer than that of the optical antipode. A stereoisomerically pure compound is generally made up of at least 90% of the desired isomer based upon 100% total weight of the compound, preferably at least 95%, preferably at least 98%, preferably at least 99%.

One advantage of the present invention is the use of commercially available thioureas, such as thiourea (III), which are safe and convenient to handle on a commercial scale. The use of this type of demethylation reagent has significant advantages with respect to the scaling up of the process to provide commercial sized batches of ODV. In addition, there are further improvements in yield and purity over prior art processes, and the raw material and reaction mixture are easy to handle and are very compatible with the reaction vessel.

Another advantage of the present invention is the use of an economic and commercially available base to facilitate the demethylation reaction. The reaction can easily be carried out using a suitable base, such as potassium hydroxide or sodium methoxide, which are relatively safe and common bases.

As discussed above, the present invention provides a novel process for the preparation of highly pure ODV free base. The process has commercial viability for production with a high degree of consistency in quality and yield of product. The ODV base prepared by the process of the present invention can subsequently be converted into pharmaceutically acceptable salts, such as the succinate or fumarate salts, for finished dosage form preparation.

A further advantage of the present invention is the improved process involving preparation of the thiourea anion in the same reaction solvent that is used to perform the demethylation. This offers a significant advantage by way of using one solvent for the whole sequence. Conversely, in the process described in U.S. Pat. No. 6,689,912 for demethylation of venlafaxine, the sodium salt of dodecane thiolate is prepared in methanol followed by further treatment with venlafaxine in polyethylene glycol 400. To drive the reaction to completion, methanol needs to be distilled off. This cumbersome procedure is avoided with the present invention.

Moreover, the present invention provides a process for the preparation of ODV base wherein the reaction can be performed at a temperature between 160° C. to 180° C. It can be achieved on a commercial scale and affords less impurities in the finished product compared with other processes reported in the prior art, where temperatures of around 190° C. and above were required.

Yet another advantage of preferred aspects of the present invention is the improved process for preparation, isolation and purification of the ODV base in high yield, with approximately 70-80% molar yield with high purity conforming to ICH guidelines of impurity profile. The processes of the present invention are capable of providing ODV base in consistent chemical purity irrespective of the scale of preparation.

In addition, the present invention offers a simple work up procedure with improved yield and quality with minimum contamination with process impurities. Therefore the process of the present invention is amenable to large scale production wherein reaction conditions can be easily controlled. Additionally, the product obtained by following the process disclosed here is readily filtered and easily dried.

The present invention further provides a process for the preparation of ODV base by reacting an anion of a thiourea with venlafaxine base or a salt of venlafaxine in a suitable solvent at relatively lower temperatures than those reported in the prior art for similar methods.

A further advantage of the present invention is that the demethylation reaction can be performed on the venlafaxine hydrochloride as well as venlafaxine free base.

Preferably the process of the present invention is performed in the presence of a protic or aprotic solvent and, optionally, a base such as a hydroxide, carbonate or alkoxide are used to generate the thiourea anion. The hydroxide is preferably comprised of monovalent or bivalent metal hydroxides such as lithium, sodium, potassium, calcium and magnesium hydroxides. Alternatively, metal carbonates or metal hydrogen carbonates can be used. The metal alkoxide is preferably comprised of a straight- or branched-chain alkyl group of 1 to 6 carbon atoms and is most preferably sodium methoxide. Organic bases such as aromatic and aliphatic amines or a salt of ammonia or an alkylammonia may also be used. The thiourea anion is preferably prepared in situ in the same solvent used for running the reaction.

The thiourea reagent (IV) is preferably a low molecular weight derivative.

The solvent used in the reaction mixture is preferably an alcoholic or ethereal solvent, more preferably an alcohol such as 1-butanol. Other preferred solvents are methyl cellosolve, ethyl cellosolve or polyethylene glycol. Preferably the solvent is an inert, polar, high boiling solvent, most preferably polyethylene glycol 400.

Preferably the crude ODV base formed by the process according to the first aspect of the invention, is purified by mixing with an alcohol, such as methanol, ethanol or isopropanol or a mixture thereof, to form a suspension and then adding acid followed by base to generate ODV base with high purity. Most preferably, the alcohol is methanol. Preferably the acid used is an inorganic acid such as hydrochloric acid or sulphuric acid. Preferably the base used is an organic base such as triethylamine or trimethylamine. Alternatively, the base used can be an inorganic base such as ammonia, sodium carbonate, potassium carbonate or sodium hydroxide.

Alternatively or additionally, purification of the crude ODV base is preferably carried out by forming a solution of the crude ODV in a straight- and branched-chain alcohol having 1 to 4 carbon atoms, preferably methanol or isopropanol, by heating at reflux and then cooling the solution, preferably to about 10° C. to 15° C., to afford pure recrystallized ODV. The product is then easily filtered to yield ODV base with high purity conforming to ICH guidelines.

In a preferred embodiment of the present invention there is provided a process for the preparation of ODV base or a salt such as a pharmaceutically acceptable salt thereof, comprising:

(a) reacting a thiourea with an appropriate base to form an anion in polyethylene glycol 400;
(b) reacting the anion with venlafaxine free base in polyethylene glycol 400 at a temperature in the range of 170-180° C.;
(c) acidifying the reaction mixture and washing the acidified reaction mixture with dichloromethane to remove impurities;
(d) isolating crude ODV base at pH>9.5; and
(e) purifying the crude ODV base either by forming a suspension of the crude ODV base in methanol and adding aqueous hydrochloric acid followed by aqueous ammonia or by crystallization of the crude ODV base in methanol or isopropanol to obtain pure ODV base.

The demethylation reagent used in the above preferred embodiment is most preferably thiourea and the base is preferably potassium hydroxide or sodium methoxide. The use of this combination of reagents is very safe and efficient on a commercial scale. Thiourea is a solid compound which is much less noxious than other sulphur reagents such as 1,2-ethane dithiol. Surprisingly this combination of reagents also synergistically affords a very pure product in high yield.

In an alternative preferred embodiment of the present invention there is provided a process for the preparation of ODV base or a salt such as a pharmaceutically acceptable salt thereof, comprising:

(a) reacting thiourea with an appropriate base (such as a hydroxide or alkoxide) to form an anion in polyethylene glycol 400;
(b) reacting the anion with venlafaxine free base in polyethylene glycol 400 at a temperature in the range of 170-180° C.;
(c) diluting the reaction mixture, acidifying the reaction mixture with hydrochloric acid to pH<4.0 and washing the acidified reaction mixture with dichloromethane to remove impurities;
(d) isolating crude ODV base at pH>9.5 by basifying the reaction mixture with ammonia solution; and
(e) crystallization of the crude ODV base in methanol

The present invention further provides a pharmaceutical composition comprising the ODV, or a pharmaceutically acceptable salt thereof, which has been prepared in accordance with the first aspect of the invention. It also provides for the use of the aforesaid pharmaceutical composition for the preparation of a medicament for the treatment of depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's Disease.

The dosage form can be a solution or suspension form, but is preferably solid and comprises one or more conventional pharmaceutically acceptable excipient(s). Preferred dosage forms in accordance with the invention include tablets, capsules and the like. Tablets can be prepared by conventional techniques, including direct compression, wet granulation and dry granulation. Capsules are generally formed from a gelatin material and can include a conventionally prepared granulate of excipients and the ODV, or a pharmaceutically acceptable salt thereof, in accordance with the invention.

For the avoidance of doubt, insofar as is practicable any embodiment of a given aspect of the present invention may occur in combination with any other embodiment of the same aspect of the present invention. In addition, insofar as is practicable it is to be understood that any preferred or optional embodiment of any aspect of the present invention should also be considered as a preferred or optional embodiment of any other aspect of the present invention.

The details of the invention, its objects and advantages are explained hereunder in greater detail in the following non-limiting examples.

### EXAMPLES

### Example 1

### Preparation of ODV Base from Venlafaxine Base Using Thiourea

Thiourea (13.7 g, 0.18 mol) was added to a suspension of potassium hydroxide (20.2 g, 0.36 mol) in polyethylene glycol 400 (50 mL) at 25-30° C. To this stirred suspension, venlafaxine base (10 g, 0.04 mol) was added and the reaction mixture was heated to 170-180° C. After completion of the reaction (16-20 hours), the reaction mixture was allowed to cool to 60-70° C. and water (40 mL) was added followed by addition of 35% aqueous hydrochloric acid (15-20 mL). The solution was washed with dichloromethane (2×50 mL). To the aqueous solution, 25% aqueous ammonia solution was added to adjust the pH of the solution to >9.5. A solid precipitated out and was filtered to afford crude ODV base. The crude ODV base was further taken in methanol (250 mL), refluxed for 1 hour and then cooled to 10-15° C. The crystallized pure ODV base was filtered and dried at 50-55° C. Instead of this crystallization in methanol, the crude ODV base could have been purified by acid/base purification in methanol by adding aqueous hydrochloric acid to a methanolic suspension and re-precipitation by adding aqueous ammonia. The product was confirmed as being ODV base on the basis of ¹H-NMR.

Weight of the product=7.6 g
Molar yield=80%
Chemical purity>99.9% (measured by HPLC)

### Example 2

### Preparation of ODV Base from Venlafaxine Hydrochloride Using Thiourea

Thiourea (12.1 g, 0.16 mol) was added to a suspension of potassium hydroxide (17.8 g, 0.32 mol) in polyethylene glycol 400 (50 mL) at 25-30° C. To this stirred suspension, venlafaxine hydrochloride (10.0 g, 0.03 mol) was added and the reaction mixture was heated to 170-180° C. After completion of the reaction (16-20 hours), the reaction mixture was allowed to cool to 60-70° C. and water (40 mL) was added followed by addition of 35% aqueous hydrochloric acid (15-20 mL). The solution was washed with dichloromethane (2×50 mL). To the aqueous solution, 25% aqueous ammonia solution was added to adjust the pH of the solution to >9.5. A solid precipitated out and was filtered to afford crude ODV base. The crude ODV base was crystallized in methanol and dried to afford pure ODV base as an off-white solid. Instead of this crystallization in methanol, the crude ODV base could have been purified by acid/base purification in methanol by adding aqueous hydrochloric acid to a methanolic suspension and re-precipitation by adding aqueous ammonia. The structure of the product was confirmed on the basis of ¹H-NMR.

Weight of the product=5.9 g
Molar yield=70%
Chemical purity>99.9% (measured by HPLC)

The ODV base prepared in either example 1 or 2 could be readily converted into a salt, such as the succinate, fumarate or hydrochloride salt, by standard techniques well known to the skilled person.

It will be understood that the present invention has been described above by way of example only. The examples are not intended to limit the scope of the invention. Various modifications and embodiments can be made without departing from the scope and spirit of the invention, which is defined by the following claims only.

1.-43. (canceled)

44. A process for the preparation of O-desmethylvenlafaxine (ODV, II), or a salt including a pharmaceutically acceptable salt thereof, comprising the reaction of venlafaxine, or a salt thereof, with a thiourea or a mixture of thioureas.

45. A process according to claim 44, wherein the thiourea is thiourea (III),

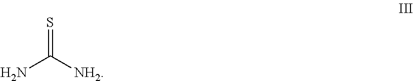

46. A process according to claim 44, wherein the thiourea is a substituted thiourea (IV), wherein R¹, R², R³ and R⁴ are independently hydrogen, alkyl, alkenyl, alkynyl, aryl, arylalkyl, arylalkenyl, arylalkynyl, alkylaryl, alkenylaryl or alkynylaryl,

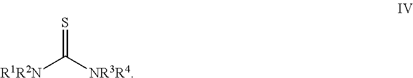

47. A process according to claim 45, wherein at least one of R¹, R², R³ or R⁴ is hydrogen.

48. A process according to claim 44, wherein:
(i) a reaction solvent is employed; and/or
(ii) a reaction solvent is employed selected from an alcohol, ethylene glycol, an ether of ethylene glycol or a mixture thereof; and/or
(iii) a reaction solvent is employed selected from polyethylene glycol (e.g. polyethylene glycol 400), cellosolve or 1-butanol.

49. A process according to claim 44, wherein a thiourea anion is generated by treatment of the thiourea with:
(i) a base; and/or
(ii) a base, wherein the base is a monovalent or bivalent metal hydroxide, carbonate, hydrogen carbonate or alkoxide; and/or
(iii) a base, wherein the base is a metal hydroxide or metal alkoxide; and/or
(iv) a base, wherein the base is potassium hydroxide or sodium methoxide; and/or
(v) a base, wherein the base is an organic base; and/or
(vi) a base, wherein the base is an alkyl or aryl amine; and/or
(vii) a base, wherein the base is piperidine or pyridine.

50. A process according to claim 44, wherein the reaction is performed at a temperature within the range of:
(i) 100-220° C.; and/or
(ii) 160-190° C.; and/or
(iii) 160-180° C.

51. A process according to claim 44, wherein during the work up procedure, to remove process impurities the reaction mixture is washed with:
(i) a solvent that is immiscible with water at 20° C. and 1 atmosphere pressure; and/or
(ii) a hydrocarbon solvent or a halogenated hydrocarbon solvent; and/or
(iii) a hydrocarbon solvent selected from cyclohexane, toluene, xylene or mixtures thereof; and/or
(iv) a halogenated hydrocarbon solvent selected from dichloromethane, ethylene dichloride or mixtures thereof.

52. A process according to claim 44, wherein the crude ODV base formed is purified by crystallization with:
(i) an alcohol to generate ODV base with high purity; and/or
(ii) an alcohol selected from methanol, ethanol, isopropanol or a mixture thereof to generate ODV base with high purity.

53. A process according to claim 44, wherein the crude ODV base formed is purified by mixing with an alcohol to form a suspension and then adding acid followed by base to generate ODV base with high purity.

54. A process according to claim 53, wherein:
(i) the alcohol is selected from methanol, ethanol, isopropanol or a mixture thereof; and/or
(ii) the alcohol is methanol; and/or
(iii) the acid used is an inorganic acid including hydrochloric acid or sulphuric acid; and/or
(iv) the base used is an organic base including triethylamine or trimethylamine, or an inorganic base including ammonia, sodium carbonate, potassium carbonate or sodium hydroxide.

55. A process according to claim 44, wherein:
(i) the pharmaceutically acceptable salt of ODV prepared is selected from the succinate or fumarate salt; and/or
(ii) the salt of venlafaxine used is the hydrochloride salt.

56. ODV or a salt including a pharmaceutically acceptable salt thereof prepared by a process according to claim 44.

57. ODV succinate or ODV fumarate prepared by a process according to claim 44.

58. A pharmaceutical composition comprising ODV or a pharmaceutically acceptable salt thereof prepared by a process according to claim 44.

59. A pharmaceutical composition comprising ODV succinate or ODV fumarate prepared by a process according to claim 44.

60. A method of treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's Disease, the method comprising administering to a patient in need thereof a therapeutically or prophylactically effective amount of ODV or a pharmaceutically acceptable salt thereof prepared by a process according to claim 44.

61. A method of treating or preventing depression, anxiety, panic disorder, generalized anxiety disorder, post traumatic stress disorder, premenstrual dysphoric disorder, fibromyalgia, agoraphobia, attention deficit disorder, social anxiety disorder, autism, schizophrenia, obesity, anorexia nervosa, bulimia nervosa, vasomotor flushing, cocaine or alcohol addiction, sexual dysfunction, borderline personality disorder, chronic fatigue syndrome, urinary incontinence or Parkinson's Disease, the method comprising administering to a patient in need thereof a therapeutically or prophylactically effective amount of ODV succinate or ODV fumarate prepared by a process according to claim 44.

62. A method according to claim 60, wherein the patient is a mammal and/or a human.

63. A method according to claim 61, wherein the patient is a mammal and/or a human.
